(19) United States
(12) Patent Application Publication
(10) Pub. No.: US 2024/0157066 A1
(43) Pub. Date: May 16, 2024

(54) INHALER

(71) Applicant: Alveon GmbH, Freienbach (CH)

(72) Inventors: André Geilen, Freienbach (CH); Ralf Martin Riess, Freienbach (CH); Simon Geiss, Freienbach (CH)

(21) Appl. No.: 18/282,457

(22) PCT Filed: Mar. 16, 2022
(86) PCT No.: PCT/EP2022/056788

§ 371 Date: Sep. 15, 2023

(30) Foreign Application Priority Data
Mar. 16, 2021 (DE) 102021202546.3

(51) Int. Cl. A61M 11/04; A61M 15/00

(52) U.S. Cl. CPC A61M 11/042; A61M 15/0001; A61M 2205/0211; A61M 2205/0233; A61M 2205/3368; A61M 2205/3653; A61M 2205/8206; A61M 2209/045

(57) ABSTRACT

An inhaler for inhaling an aerosol may include a container configured to store a liquid, an evaporator device, and a controller. The evaporator device may include (i) an electric evaporator configured to evaporate the liquid to produce the aerosol and (ii) a receiving structure having a predetermined total volume for receiving the liquid. The inhaler may be adjustable to a filling state and to an evaporation state. In the filling state, the receiving structure and the container may be fluidically connected such that the liquid stored in the container is flowable into the receiving structure. In the evaporation state, the receiving structure and the container may be fluidically separated such that the liquid is not flowable from the container into the receiving structure. The controller may be configured to electrically supply the evaporator in the evaporation state such that the liquid received in the receiving structure evaporates.

### CROSS-REFERENCE TO RELATED APPLICATIONS

This application claims priority to International Patent Application No. PCT/EP2022/056788, filed on Mar. 16, 2022, and German Patent Application No. DE 10 2021 202 546.3, filed on Mar. 16, 2021, the contents of both of which are hereby incorporated by reference in their entirety.

### TECHNICAL FIELD

The present invention relates to an inhaler for inhaling an aerosol, which comprises a container for storing a liquid provided for evaporation and an evaporator device for evaporating the liquid and thus producing the aerosol. Further, the invention relates to a unit for such an inhaler which includes the evaporator device and the container.

### BACKGROUND

An inhaler is used for producing an aerosol for inhaling. For this purpose, a liquid is generally evaporated in an evaporator device and an aerosol thus produced and dispensed. Usually, the evaporator device includes a receiving structure for receiving the liquid to be evaporated and an electric evaporator for evaporating the liquid.

From DE 10 2016 120 803 A1 an inhaler having an evaporator device is known. The evaporator device comprises an evaporator of a doped and electrically conductive ceramic. The ceramic is provided with controlled micro-channels having a predetermined orientation, through which liquid flows for the purpose of evaporation. Upon electrical supply, the ceramic generates heat in order to evaporate the liquid received therein. Further, the evaporator device comprises a flow control device, which controls the flow of the liquid through the micro-channels.

From WO 2004 022 242 A1 an inhaler is known which comprises an evaporator device having a heated mono-capillary. The inhaler, further, comprises a container for storing a liquid which can flow to the mono-capillary. The flow of the liquid to the mono-capillary is controlled via a valve.

From US 20 2000 022 416 A1 an inhaler is known, which comprises a container for storing a liquid. In the container, a piston is guided which upon adjustment applies liquid from the container to an evaporator. During the operation, the evaporator generates heat in order to evaporate the liquid applied to the evaporator.

### SUMMARY

The present invention deals with the object of stating for an inhaler of the type mentioned at the outset and for a unit for such an inhaler, improved or at least other embodiments, which are characterised in particular by an improved control of the evaporated liquid.

According to the invention, this object is solved through the subject matter of the independent claim(s). Advantageous embodiments are the subject matter of the dependent claim(s).

The present invention is based on the general idea of providing an inhaler having an evaporator device, which comprises a receiving structure with a predetermined total volume for receiving a liquid to be evaporated, wherein the receiving structure is filled with liquid by means of a fluidic connection with a container for storing the liquid and subsequently fluidically separated from the container, and wherein the liquid received in the receiving structure, in the state of the receiving structures fluidically separated from the container, is evaporated for producing the aerosol. The predetermined and thus known total volume of the receiving structure results in that during the evaporation of the liquid received in the receiving structure the quantity or at least maximally possible quantity of the evaporated liquid is known and thus precisely controllable. This results in an improved control of the quantity of the liquid evaporated in each case during the evaporation and thus in an improved control of the aerosol dispensed for inhalation. On the whole, an improved control over the evaporation process and the evaporated quantity is thus achieved. Thus it is possible in particular to employ the inhaler for medicinal application.

According to the inventive idea, the inhaler comprises the container for storing a liquid and the evaporator device. The evaporator device includes the receiving structure and an electric evaporator for evaporating the liquid and thus producing the aerosol. The receiving structure comprises a predetermined and thus known total volume for receiving the liquid. The inhaler is adjustable between a filling state and an evaporation state. In the filling state, the receiving structure and the connection are fluidically connected to one another so that liquid stored in the container enters the receiving structure. In the evaporation state, the receiving structure and the container, by contrast, are fluidically separated from one another so that a flow of liquid from the container into the receiving structure is prevented. Liquid received in the receiving structure in the evaporation state is evaporated by means of the electric evaporator for producing the aerosol. For this purpose, the inhaler comprises a suitably configured control device. The control device is consequently configured in such a manner that it electrically supplies the evaporator in the evaporation state so that liquid received in the receiving structure, evaporates.

Practically, the evaporator is configured in such a manner that in the evaporation state, upon electrical supply, it generates heat for evaporating the liquid received in the receiving structure. In particular, the evaporator can be designed as a heating resistor or comprise a heating resistor.

Preferably, the inhaler is a mobile and manually portable inhaler which can thus be carried along. Preferably, the inhaler can be manually gripped by a user and carried. The inhaler is designed accordingly with respect to its dimensions.

Preferably, the inhaler comprises a battery, preferentially a rechargeable battery for the electrical supply. The control device is practically configured in such a manner that by means of the battery it electrically supplies the evaporator in the evaporation state for evaporating the liquid received in the receiving structure.

Basically, the inhaler can be employed for evaporating any liquids. In particular it is possible to employ the inhaler for evaporating liquids containing medical substances. In particular, the defined and/or controllable quantity of the evaporated liquid allows a defined and/or controlled dosing of the active substance administered to a patient.

In preferred embodiments, a complete filling of the receiving structure with the liquid stored in the container takes place in the filling state. This results in an improved control of the quantity of the evaporated liquid. Basically, the complete filling of the receiving structure can be realised by the inhaler remaining long enough in the filling state. Alternatively or additionally it is conceivable to employ the control device for realising the complete filling of the receiving structure. For this purpose, the control device is suitably configured.

Preferred are embodiments, in which the evaporation of the liquid in the receiving structure merely takes place outside of the filling state and in the evaporating state. Thus, there is an improved control of the quantity of the evaporated liquid. Thus, the inhaler is operated discontinuously because the evaporation device, after the evaporation of the liquid received in the receiving structure, can no longer produce vapour. Only in the filling state does liquid again enter the receiving structure and can be evaporated with the evaporator. For this purpose it is preferred when an electrical supply of the evaporator is interrupted in the filling state. For this purpose, the control device is advantageously configured in such a manner that it interrupts the electrical supply of the evaporator in the filling state.

A continuous operation of the inhaler is also conceivable, during which the evaporator in the filling state evaporates liquid received in the receiving structure, wherein liquid continuously re-enters the receiving structure.

In preferred embodiments, a refilling of liquid in the receiving structure takes place only in particular when the liquid previously received in the receiving structure is completely evaporated. This makes possible to easily and reliably control the evaporated quantity of the liquid. For this purpose, the control device can be suitably configured. Thus, the control device can be configured so that it prevents an adjusting from the evaporating state into the filling state until the liquid received in the receiving structure is completely evaporated. The evaporation of the liquid received in the receiving structure can take place by way of one evaporation operation or by multiple evaporation operations.

Alternatively it is conceivable to monitor the quantity of the liquid evaporated during the respective evaporation operation by means of suitable sensorics.

The evaporation of the liquid received in the receiving structure takes place at sufficiently high temperatures of the receiving structure. The evaporator device, in particular the evaporator is configured accordingly. This means that the evaporator upon electrical supply generates heat of sufficiently high temperatures which result in the evaporation of the liquid received in the receiving structure.

A complete evaporation of the liquid received in the receiving structure takes place in particular when the receiving structure is correspondingly heated for a predetermined and thus known duration. In other words, a complete evaporation of the liquid received in the receiving structure is achieved in particular when the evaporator is operated for a predetermined duration and/or produces for a predetermined duration temperatures in a predetermined range, in the following also referred to as operating range. The complete evaporation of the liquid received in the receiving structure can be controlled and/or monitored by means of the control device. Thus, the control device is configured accordingly. In particular it is sufficient when the control device controls and/or monitors the duration of the operation of the evaporator.

Basically, the receiving structure and the evaporator of the evaporator device can be separate constituent parts of the evaporator device.

Embodiments are considered advantageous, in which the receiving structure is at least partly, preferentially completely part of the evaporator. This means that the evaporator comprises the receiving structure at least partly, preferentially completely. By way of this, the receiving of the liquid and the evaporating of the liquid take place in the evaporator. In other words, both the heat generation for evaporating the liquid and the receiving of the liquid take place in the evaporator. Thus, the receiving structure during the operation has a homogeneous temperature so that the liquid received in the receiving structure is homogeneously and evenly heated and evaporated.

In addition, the temperature of the evaporator thus corresponds to the temperature of the receiving structure. This results in a simplified control of the evaporated quantity of the liquid and to an improved efficiency of the inhaler.

Preferred are embodiments, in which the evaporator is designed as an electrically conductive ceramic comprising the receiving structure, in the following also referred to as evaporator ceramic.

Thus, advantageously, the evaporator ceramic also serves simultaneously for receiving and storing the liquid to be evaporated and generating heat for evaporating the liquid. The storing of the liquid takes place by means of the receiving structure of the evaporator ceramic. Thus, the evaporator ceramic comprises the receiving structure in which during the operation the liquid to be evaporated is received.

For the electrical supply of the evaporator, in particular of the evaporator ceramic, the evaporator device comprises two electrical connections. A path of the electric current, in the following also referred to as “current path”, runs between the two electrical connections and through the evaporator, in particular through the evaporator ceramic.

Preferably, the evaporator, in particular the evaporator ceramic, is configured in such a manner that during the operation upon electrical supply it homogeneously generates heat in the evaporator, in particular in the evaporator ceramic by means of its electrically conductive characteristic, in order to evaporate the liquid received in the receiving structure. The homogeneous heat generation of the evaporator ceramic results in a homogeneous temperature or a homogeneous heat distribution in the volume of the evaporator ceramic and consequently in the receiving structure. This results in an even evaporation of the liquid in the entire receiving structure.

The evaporator, in particular the evaporator ceramic, is designed for the operation in a thermal operating range which is delimited by a lower operation starting temperature and an upper operation end temperature. In other words, for evaporating the liquid, the evaporator, in particular the evaporator ceramic, upon electrical supply, generates heat in the operating range and thus between the operation starting temperature and the operation end temperature.

The control of the temperature of the evaporator, in particular of the evaporator ceramic, can basically take place by means of suitable sensorics which, practically, are communicatingly connected to the control device.

Embodiments are considered advantageous, in which the inhaler, in particular the evaporator device, comprises an electrical conductor which in the following is also referred to as blocking conductor. The blocking conductor is arranged in the current path so that the electric current during the operation flows through the blocking conductor. The blocking conductor is heat-transmittingly connected to the evaporator, in particular the evaporator ceramic. In addition, the blocking conductor is configured in such a manner that, at the operation end temperature, it has an abruptly increasing electrical resistance. Because of the heat-transmitting connection of the blocking conductor with the evaporator ceramic, an abrupt increase of the electrical resistance in the current path and thus an interruption or at least substantial reduction of the electrical supply of the evaporator, in particular of the evaporator ceramic consequently takes place when the operation end temperature is reached. Accordingly, the operation end temperature is at least substantially influenced, preferentially determined by means of the blocking conductor.

Thus, the operation end temperature is predetermined and determined without additional sensorics. Upon electrical supply of the evaporator, in particular of the evaporator ceramic, the evaporator thus has a temperature between the operation starting temperature and the operation end temperature. Thus, the quantity of the evaporated liquid can be controlled in a simple and reliable manner via the duration of the electrical supply of the evaporator. In particular it is possible in this manner to achieve the complete evaporation of the entire liquid received in the receiving structure via a predetermined duration of the electrical supply of the evaporator.

The heat-transmitting connection of the blocking conductor with the evaporator, in particular the evaporator ceramic, is advantageously such that the temperature of the blocking conductor corresponds at least substantially to the temperature of the evaporator, in particular of the evaporator ceramic.

The inhaler, in particular the evaporator device, can basically comprise a single blocking conductor.

It is also conceivable to provide the inhaler, in particular the evaporator device, with two or more such blocking conductor. Here it is preferred when the blocking conductors are identical.

Here, an abrupt increase of the electrical resistance upon exceeding of the operation end temperature is to mean an increase which exceeds a linear increase.

It is preferred when at least one of the at least one blocking conductors, upon exceeding the operation end temperature, shows a potential increase of the electrical resistance. Particularly preferred are embodiments, in which at least one of the at least one blocking conductors is configured in such a manner that its electrical resistance, upon exceeding the operation end temperature, starts to increase exponentially. Embodiments prove to be advantageous, in which the electrical resistance of at least one of the at least one blocking conductors, advantageously of the respective blocking conductor, rises by at least one power of ten in the 50° C. following the operation end temperature. Thus, the evaporation parameters can be particularly easily and effectively controlled.

Embodiments are considered advantageous, in which at least one of the at least one blocking conductors, advantageously the respective blocking conductor, is configured as a PTC thermistor, wherein the operation end temperature is between a starting temperature and an end temperature of the at least one PTC thermistor. PTC thermistors have a characteristic current characteristic, wherein the electrical resistance from the starting temperature abruptly increases by multiple powers of ten. It is thus achieved that the blocking conductor up to the starting temperature does not or only marginally influence the electrical resistance of the entire evaporator device, in the following also referred to as total resistance, and that the blocking conductor has an influence increasing the total resistance only when the starting temperature is reached. In other words, the total resistance up to the reaching of the operation end temperature is dominated by the evaporator, in particular by the evaporator ceramic, and upon reaching the operation end temperature, by the at least one blocking conductor. As a consequence, the operation in the thermal operating range can take place with reduced energy expenditure and thus higher efficiency. At the same time, a precisely defined and reliable interruption or at least reduction of the electrical supply takes place when the operation end temperature is reached.

Basically, the operation end temperature can be anywhere between the starting temperature and the end temperature, advantageously between the starting temperature and the rated temperature of the PTC thermistor.

It is advantageous when the operation end temperature corresponds to the starting temperature of the PTC thermistor. Thus it is achieved, in particular, that for reaching the operation end temperature no increased energy expenditure, in particular no increased current consumption is necessary. This results in an increased efficiency of the evaporator device. In addition, the evaporator device can thus be operated easily and for an increased operating duration with batteries, in particular rechargeable batteries. Further, this results in that the blocking conductor in the operating range produces no or preferably no heat. There is thus an improved control over the evaporation temperature. Further it is thus possible to operate the evaporator, in particular the evaporator ceramic with a power which, without the blocking conductor, would result in overheating. Because of this it is possible without elaborate control to bring the evaporator, in particular the evaporator ceramic quickly and with high power up to temperatures in the operating range and keep these in the operating range.

Basically, the respective at least one blocking conductor can be freely arranged in the current path, provided it is connected to the evaporator, in particular the evaporator ceramic so as to transmit heat.

It is conceivable in particular to arrange at least one of the at least one blocking conductors between the evaporator and one of the connections. This allows a simple and compact design of the evaporator device.

Basically, the heat-transmitting connection between the respective blocking conductor and the evaporator, in particular the evaporator ceramic, can be freely configured.

Preferred are embodiments, in which at least one of the at least one blocking conductors, advantageously the respective blocking conductor, lies flat on the evaporator, in particular the evaporator ceramic. In particular, one of the at least one blocking conductors can lie directly flat on the evaporator, in particular the evaporator ceramic. This results in a simple and compact design of the evaporator device, wherein at the same time a simple and reliable heat transmission from the evaporator, in particular from the evaporator ceramic, to the blocking conductor is present. At the same time, it is thus easily possible to arrange the blocking conductor in the current path.

The evaporator, in particular the evaporator ceramic, is advantageously formed integrally and contiguously. Here it is preferred when on at least one outer side of the evaporator, in particular of the evaporator ceramic, a blocking conductor is arranged. Thus, a compact and simple manufacture and design of the inhaler is possible.

It is also conceivable to configure the evaporator, in particular the evaporator ceramic, in two or multiple parts. The evaporator, in particular the evaporator ceramic, can thus comprise two evaporator bodies that are separate from one another. Between at least two of the at least two evaporator bodies, a blocking conductor can be arranged.

Preferred are embodiments, in which the evaporator consists of the evaporator ceramic, i.e. exclusively comprises the evaporator ceramic. This results in a simplified manufacture of the evaporator device and at the same time a more precise and/or simple control of the evaporation parameters, in particular of the total volume for receiving the liquid to be evaporated and of the generated heat.

The respective at least one blocking conductor can basically be produced from any material provided it has an abruptly increasing electrical resistance when exceeding the operation end temperature.

It is conceivable, in particular, that at least one of the at least one blocking conductors is a ceramic.

Practically, the at least one blocking conductor is dimensioned in such a manner that, compared with the evaporator, in particular the evaporator ceramic, it makes up a smaller proportion in terms of volume. This allows in particular a more compact configuration of the evaporator device.

Preferred are embodiments, in which at least one of the at least one blocking conductors is formed as a layer. In particular, compared with the evaporator, in particular the evaporator ceramic, the at least one blocking conductor thus has a substantially reduced volume.

The respective layer can basically be freely configured. In particular, at least one of the at least one layer can be configured as a film, a coating and the like.

As described above it is preferred when the electric total resistance of the evaporator device is dominated in the thermal operating range by the evaporator, in particular the evaporator ceramic, and above the operating range, i.e. when exceeding the operation end temperature, by the blocking conductor.

Preferably this is realised in such a manner that the electrical resistance of the blocking conductor in the operating range corresponds maximally to half of the electrical resistance of the evaporator, in particular of the evaporator ceramic.

The electrical resistance of the at least one blocking conductor is composed in particular of the specific resistance and the volume or the distance along the current path. Accordingly, a reduction of the electrical resistance of the blocking conductor in the operating range can be achieved by a reduction of the relative volume of the blocking conductor in the inhaler, in particular in the evaporator device.

The evaporator, in particular the evaporator ceramic, preferentially comprises an electrical resistance which, up to the operation end temperature, in particular compared with the increase of the resistance of the blocking conductor from the operation end temperature, increases marginally. Preferably, the electrical resistance of the evaporator, in particular of the evaporator ceramic, follows in the operating range a temperature-dependent course in such a manner that the resistance increases with the temperature maximally by a power of ten.

As already mentioned, the evaporator ceramic, upon electrical supply, homogeneously generates heat by means of its electrical conductivity. In particular, the evaporator ceramic is a heating resistor.

The evaporator ceramic can be an electrically conductive ceramic of any kind, provided it has the receiving structure and upon electrical supply, in particular upon applying an electrical voltage in a predetermined range, homogeneously generates heat in the operating range.

It is conceivable that the evaporator ceramic per se is electrically conductive. Among these are for example ceramics of metal oxides, such as titanium oxide or metal carbides such as silicon carbide. Likewise, composite ceramics can be employed which comprise electrically conductive and electrically non-conductive networks of different materials, wherein the conductive networks are practically distributed homogeneously in the ceramic. Examples for such composite ceramics are such having metal oxides of different oxidation stage. Mixed oxide ceramics can also be employed which are manufactured by mixing different starting materials, wherein for manufacturing the ceramic, typically during the sintering, a new material is created through chemical reactions. Examples for the starting materials are different metal oxides. Further, doped ceramics can be employed which become electrically conductive by the doping. Obviously, any combinations of the mentioned ceramics can also be employed provided the evaporator ceramic is an electrically conductive ceramic with the receiving structure, which during the operation homogeneously generates heat.

Basically, for adjusting the inhaler between the filling state and the evaporation state, a valve device having at least one valve can be provided, which suitably establishes and cuts the fluidic connection between the container and the receiving structure.

In preferred embodiments, the adjusting of the inhaler between the filling state and the evaporation state takes place by a relative movement between the container and the evaporator device, in particular the receiving structure. The respective state thus corresponds to a relative position between the container and the receiving structure. In other words, the filling state corresponds to a filling position and the evaporation state to an evaporation position. It is thus possible, in particular, to do without a corresponding valve device. The inhaler can thus be manufactured more easily and cost-effectively as well as reduced in weight.

Preferred are embodiments, in which the container comprises a container outlet for letting out the liquid into the receiving structure and thus filling the receiving structure with the liquid. In addition, the receiving structure comprises an outer surface which in the filling state is fluidically connected to the container outlet, via which liquid via the container outlet enters the receiving structure. This outer surface is also referred to as receiving surface in the following. The evaporator device comprises a seal adjoining the receiving surface. The receiving surface in the filling state adjoins the container outlet so that the receiving structure is fluidically connected to the container. In the evaporation state, the seal, by contrast, seals the container outlet. Thus, a simple adjusting of the inhaler between the filling state and the evaporation state takes place. Further, the outflow of liquid from the container outlet in the evaporation state is thus avoided in a simple and reliable manner.

Embodiments are considered advantageous, in which the filling of the receiving structure with the liquid and the dispensing of the generated vapour from the receiving structure takes place via portions of the outer surface of the receiving structure spaced apart from one another. This means that the receiving structure comprises an outer surface that is spaced apart from the receiving surface, in the following also referred to as dispensing surface, from which during the operation upon electrical supply of the evaporator, vapour is dispensed. There is thus a simple implementation of the inhaler with simultaneously improved control of the evaporated quantity of the liquid.

Basically, the receiving surface and the output surface of the receiving structure can be freely positioned relative to one another provided they are spaced apart from one another.

It is conceivable, in particular that the receiving surface and the output surface of the receiving structure face away from one another.

Embodiments are considered advantageous, in which the container has an inner contour which comprises the container outlet. The evaporation device for adjusting between the filling state and the evaporation state is guided along the inner contour. Thus, the inhaler is configured in a simple and compact manner. In addition, the adjusting of the inhaler between the filling state and the evaporation state is thus realised in a simple manner.

It is preferred when the container is configured in the manner of a cylinder and the evaporator device, in particular the evaporator, in the manner of a piston guided in the cylinder. The cylinder practically comprises an inner surface which, jointly with an outer surface of the cylinder, delimits a volume of the container for storing the liquid, in the following also referred to as container volume. The evaporator device, in particular the evaporator, is arranged and guided outside the container volume and along the inner surface. Advantageously, the inner contour forms the inner surface at least partially. This results in a particularly simple configuration of the inhaler and a particularly simple adjustment between the filling state and the evaporation state.

It is advantageous when the inner surface of the container, in particular the inner contour, forms a chimney for the liquid evaporated with the evaporator. This means that the cross-section delimited with the inner surface decreases towards an outlet of the inhaler for dispensing the aerosol. In particular, the cross-section is constant and decreases in the evaporation state in a portion following the evaporator device. In particular, the cylinder is formed as such a chimney.

Basically, the receiving structure can be freely configured. Preferentially, the receiving structure is configured and/or formed integrally in the evaporator, in particular in the evaporator ceramic.

The receiving structure preferably distributed homogeneously, in particular homogeneously in the evaporator ceramic.

It is conceivable to introduce the receiving structure by subsequent working into the evaporator device, in particular into the evaporator ceramic. It is conceivable to form channels, in particular micro-channels in the evaporator device, in particular in the evaporator ceramic, which are part of the receiving structure or form the receiving structure.

Preferably, the receiving structure comprises pores, advantageously in the evaporator ceramic. Particularly preferably, the receiving structure consists of pores, thus is a pore structure.

The pores of the evaporator ceramic are advantageously formed during the manufacture of the evaporator ceramic, which can take place for example by means of sintering. In other words, the pores for receiving the liquid are advantageously introduced not separately, in particular not subsequently, into the evaporator ceramic. Thus, an intrinsic characteristic of the evaporator ceramic provided by the manufacture is used for storing the liquid to be evaporated. This results in a simple and cost-effective manufacture of the evaporator ceramic and thus of the evaporator device and of the inhaler.

In addition, by the manufacture of the evaporator ceramic the total volume can be defined by the pores. This results in a further easily configured control of the evaporation parameters.

Basically, the evaporator device, in particular the evaporator ceramic, can comprise pores of any kind.

It is advantageous when the evaporator ceramic comprises pores having a mean size between 0.05 μm and 50 μm. With a liquid as substance, these mean pore sizes result in a ratio between the surface and the volume of the respective pore such that these have capillary forces which offset, preferentially outweigh, the forces acting due to gravity and/or due to pressure on a drop-shaped particle of the liquid received in the volume. This results in that the drop-shape particles, in the following also referred to as droplets, remain in the pores. Consequently, a draining of the droplets and consequently of the liquid from the evaporator ceramic is prevented or at least substantially reduced. Thus, low-viscosity liquids can also be received and stored in the evaporator ceramic. Thus it is also possible with the evaporator ceramic to receive and store a greater variability of liquids of different viscosity without the liquids draining from the evaporator ceramic. As a consequence, the liquids can be provided more cost-effectively and in a wider spectrum. In particular, active substances received in the liquids can be provided more easily and/or with a more precise dosage. Thus, the evaporator ceramic and the associated evaporator device can be more easily employed for a controllable inhalation of the said active substances and thus a controllable and/or predetermined dosage of the active substances. The capillary forces described above further result in that the evaporator ceramic, when hydraulically connected to the liquid to be evaporated, saturates itself with the liquid without further action, such as for example an active pumping of the liquid into the evaporator ceramic. Altogether, separate seals of the evaporator ceramic can thus be omitted or at least reduced and/or devices for introducing the liquid into the ceramic be omitted. Thus, both the evaporator ceramic and also an associated evaporator device can be easily and cost-effectively realised. Thus, besides an increase of the possible uses of the evaporator ceramic and of the associated evaporator device, there is also a simplified implementation of the same.

A further advantage of the said mean pore sizes must be seen in that these result in an enlargement of the area of the droplets that are in contact with the evaporator ceramic. In other words, an enlarged area of the evaporator ceramic transmits heat to the droplets for evaporating the liquid. This results in a more even evaporation of the liquid and thus an improved control over the evaporation. At the same time, a more rapid evaporation of the liquid occurs in this manner.

Mean pore size, here, is to mean in particular the ratio between four times the volume and the area of the pores, i.e. 4V/A, as is stated in particular in the standard IS015901.

As described above, the evaporator ceramic can be employed in particular for receiving low-viscosity liquids. Low-viscosity liquids are in particular liquids which have a viscosity of 45 mPas and lower.

The liquid can be any liquid. In particular, it is possible to employ liquids containing medical substances.

It is preferred when the pore sizes of the pores of the pore structure at least for the greatest part are within the mean pore size. This means in particular that maximally 10% of the pores have pore sizes that are greater than 4 times the mean pore size. This results in that pores having pore sizes above the mean pore size are reduced, preferentially not present. As a consequence, the effects of pores with pore sizes above the mean pore size on the overall behaviour of the evaporator ceramic and consequently the effects of droplets with larger volumes in these pores on the overall behaviour of the liquid received in the evaporator ceramic are negligible or at least reduced. Thus, it can be prevented in particular that the liquid drains out of the evaporator ceramic. Further, this results in that the droplets received in the pores, corresponding to the size distribution of the pores, substantially same volumes. This results in a homogenous distribution of the liquid received in the evaporator ceramic via the volume of the ceramic. In addition to this, the liquid can thus be evaporated more homogenously and/or controllably.

Embodiments are considered advantageous, in which the mean pore size amounts to between 0.1 μm and 25 μm, preferably between 0.15 μm and 10 μm, particularly preferably between 0.2 μm and 5 μm. Thus, an advantageous interaction between the droplets received in the pores, the capillary forces and the distribution of the liquid in the volume of the evaporator ceramic occurs, which result in an improved reception of the liquid in the evaporator ceramic and an improved evaporation of the liquid received in the evaporator ceramic. The evaporator device and/or the container can each be a fixed part of the inhaler or be replaceably arranged in the inhaler.

It is conceivable that the evaporator device and the container are a fixed part of the inhaler, wherein the container can be refilled with liquid.

It is conceivable that the evaporator device is a fixed part of the inhaler, whereas the container is replaceably received in the inhaler.

In preferred embodiments, the container and the evaporation device form a unit replaceably received in the inhaler, in particular in the manner of a capsule. This results in a simplified handling of the inhaler. In addition, cross-contaminations of different liquids are thus avoided because in each case a container is clearly assigned to an associated evaporator device accordingly.

Here it is preferred when the container is sealed, i.e. not refillable with liquid without being destroyed. This results in particular in the avoidance of cross-contaminations of different liquids and/or prevents the use of unspecified and/or not approved liquids, or at least renders such use more difficult.

Advantageously, the control device is communicatingly connected to the evaporator device and/or with the container in such a manner that the control device is imparted and/or detects the liquid stored in the container or received in the receiving structure. Thus it is possible in particular to perform for different liquids an associated electrical supply of the evaporator device. Further it is thus possible to prevent the evaporation of non-approved and/or released liquids. Likewise it is thus possible to allow an operation of the inhaler, in particular an electrical supply of the evaporator device for evaporating the liquid, merely in particular when predetermined and/or approved containers and/or evaporation devices in received in the inhaler. The control device is configured accordingly. It is thus possible, in particular, to prevent misuse of the inhaler.

The communication between the control device and the container and/or the evaporator device is advantageously realised via corresponding communications interfaces. The control device thus comprises a control device communications interface, which is communicatingly connected to a container communications interface of the container and/or with an evaporator communications interface of the evaporator device.

The communication can take place in any way. Conceivable is a wired communication. Preferred is a wireless communication.

Preferred are embodiments, in which the unit with the container and the evaporator device comprises a common communications interface for communication with the control device communications interface.

It is to be understood that besides the inhaler, the unit including the container and the evaporator device as such are also included in the scope of this invention.

Further important features and advantages of the invention are obtained from the subclaims, from the drawings and from the associated figure description by way of the drawings.

It is to be understood that the features mentioned above and still to be explained in the following cannot only be used in the respective combination stated but also in other combinations or by themselves without leaving the scope of the present invention.

### BRIEF DESCRIPTION OF THE DRAWINGS

Preferred exemplary embodiments of the invention are shown in the drawings and are explained in more detail in the following description, wherein same reference numbers relate to same or similar or functionally same components. It shows:

FIG. 1 shows a highly simplified representation of an inhaler in the manner of a circuit diagram,

FIG. 2 shows an isometric view of an evaporator device of the inhaler,

FIG. 3 shows an isometric view of the evaporator device with another exemplary embodiment,

FIG. 4 shows a simplified section through the inhaler in a filling state,

FIG. 5 shows a simplified section through the inhaler in an evaporation state.

### DETAILED DESCRIPTION

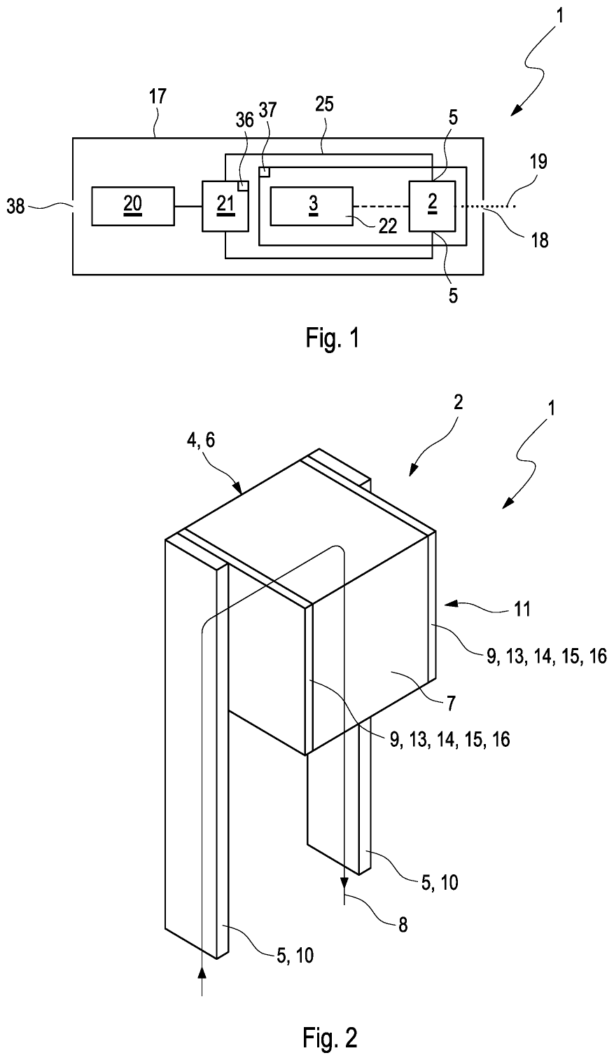

An inhaler 1, as is shown for example in the FIGS. 1 to 5, serves for inhaling an aerosol. For this purpose, the inhaler 1 comprises an evaporator device 2 and a container 3. The container 3 serves for storing a liquid to be evaporated. The evaporation of the liquid for the purpose of producing the aerosol to be inhaled takes place using the evaporator device 2.

The inhaler 1 of the shown exemplary embodiment is a mobile and manually portable inhaler 1, which when used is manually gripped and portable by a user who is not shown. With respect to its dimensions and its weight, the inhaler 1 is configured accordingly.

The evaporator device 2 serves for evaporating a liquid, in particular a predetermined dose of the liquid. The liquid is for example such a liquid which can contain a medical substance, so that upon evaporation a vapour 19 containing the active substance (see FIG. 1 and FIG. 5) is dispensed, which is inhaled by a user.

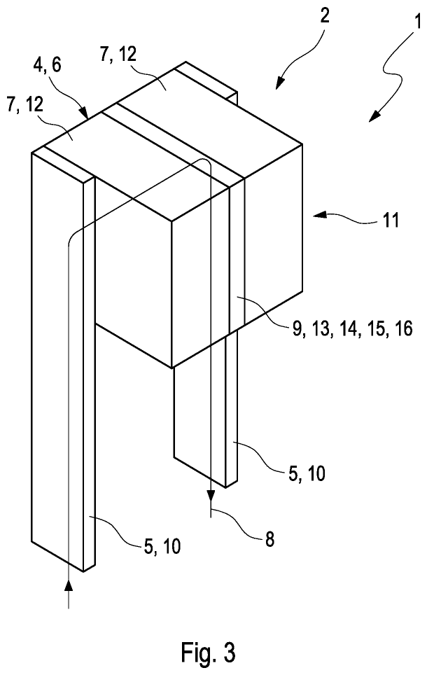

As is evident from the FIGS. 2 and 3, the evaporator device 2 comprises an electric evaporator 4 for evaporating the liquid. For the electrical supply of the evaporator 4, two electrically connections 5 are provided in the shown exemplary embodiments. Further, the evaporator device 2 comprises a receiving structure 7 for receiving liquid. The receiving structure 7 comprises a predetermined and thus known total volume for receiving liquid.

In the shown exemplary embodiments, the evaporator 4 serves both for receiving and storing the liquid to be evaporated and also for generating heat for the purpose of evaporating the liquid. For this purpose, the evaporator 4 of the shown exemplary embodiments comprises an electrically conductive ceramic 6, which in the following is also referred to as evaporator ceramic 6. In the shown exemplary embodiments, the evaporator 4 consists of the evaporator ceramic 6.

The receiving structure 7 of the exemplary embodiments shown comprises pores (not shown) of the evaporator ceramic 6, so that the liquid to be evaporated is received in the pores. Advantageously, the receiving structure 7 consists of the pores, i.e. is a pore structure. Advantageously, the receiving structure 7 thus consists of pores of the evaporator ceramic 6. The evaporator ceramic 6 can contain at least one metal oxide. For evaporating the liquid, the evaporator ceramic 6 is operated in a thermal range, which in the following is also referred to as operating range. The operating range is delimited by a low temperature, in the following also referred to as operation starting temperature and by a high temperature, in the following also referred to as operation end temperature. This means that the evaporation of the liquid to be evaporated and received in the pores takes place in the operating range and thus between the operation starting temperature and the operation end temperature.

For generating heat, the evaporator 4 is electrically supplied by means of the connections 5, so that a path 8 indicated in the FIGS. 2 and 3 of the electric current leads between the connections 5 and through the evaporator 4, wherein the path 8 is also referred to as current path 8 in the following. Upon electrical supply, the evaporator ceramic 6 generates heat for evaporating the liquid by means of its electrical resistance.

The inhaler 1 comprises a housing 17 merely shown in FIG. 1, in which the evaporator device 2 is received, and which comprises an outlet opening 18 for letting out the vapour 19 or aerosol produced by means of the evaporator 4. Further, the inhaler 1, in particular the housing 17, comprises an inlet opening 38 for letting air into the inhaler 1. In addition, the container 3 is received in the housing 17. Further, the inhaler comprises a, preferentially rechargeable battery 20, merely shown in FIG. 1, for the electrical supply of the evaporator device 2, which is received in the housing 17.

The container 17 has a volume 22 for storing liquid, which in the following is also referred to as container volume 22.

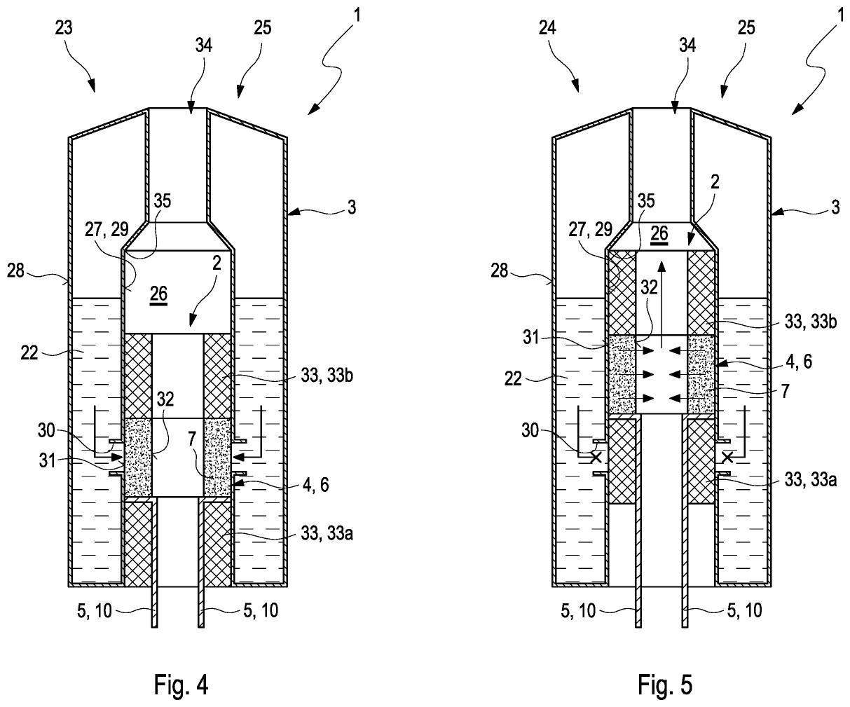

The inhaler 1 is adjustable between a filling state 23 shown in FIG. 4 and an evaporation state 24 shown in FIG. 5. In the filling state 23, the receiving structure 7 is fluidically connected to the container volume 22, so that liquid stored in the container volume 22 enters the receiving structure 7 and fills the receiving structure 7. Preferentially, a complete filling of the receiving structure 7 with the liquid takes place in the filling state 23. In the evaporation state 24, the receiving structure 7 by contrast is fluidically separated from the container volume 22, so that no liquid from the container 3 enters the receiving structure 7.

The evaporation of the liquid preferably takes place merely in the evaporation state 24. This means that the evaporator 4, in the shown exemplary embodiments the evaporator ceramic 6, merely generates heat for evaporating the liquid received in the receiving structure 7 in the evaporation state 24. For this purpose, the inhaler 1 comprises a control device 21 merely shown in FIG. 1, which is configured correspondingly. Thus, in the evaporation state 24, the control device 21 supplies the evaporator 4 electrically so that the liquid received in the receiving structure 7 evaporates. In addition, the electrical supply of the evaporator, in particular of the evaporator ceramic 6, is additionally interrupted in the filling state 23. The control device 21 is connected to the battery 20 in such a manner that it can establish and interrupt the electrical connection of the battery 20 with the evaporator device 2 for the purpose of electrically supplying the evaporator device 2.

The supply of the evaporator 4 can take place in such a manner that the liquid received in the receiving structure 7 evaporates completely. Alternatively, the supply of the evaporator 4 can take place in such a manner that a part of the liquid received in the receiving structure 7 evaporates. In this case, the liquid is evaporated in multiple steps. In each case it is preferred when the receiving structure 7, following the complete evaporation of the liquid received therein, i.e. in the dry state, is filled with liquid. This means that the inhaler 1 is adjusted or can be adjusted into the filling state 23 in particular when the liquid received in the receiving structure 7 has been evaporated completely. This can be realised by means of the control device 21, which prevents an adjusting from the evaporation state 24 into the filling state 23 until the liquid received in the receiving structure 7 is completely evaporated.

In the shown exemplary embodiments, as is evident from the FIGS. 2 and 3, at least one blocking conductor 9 is arranged in the current path 8 in such a manner that the current path 8 mandatorily leads through the blocking conductor 9. The blocking conductor 9 is merely shown and represented in the FIGS. 2 and 3. In the shown exemplary embodiments, this is achieved in that the at least one blocking conductor 9 is arranged between the connections 5. The at least one blocking conductor 9 is connected to the evaporator ceramic 6 so as to transmit heat. In the shown exemplary embodiments, the heat-transmitting connection of the at least one blocking conductor 9 to the evaporator ceramic 6 is realised by a flat arrangement of the blocking conductor 9 on the evaporator ceramic 6. In particular, the blocking conductor 9 directly lies against the evaporator ceramic 6. Thus, the temperature of the at least one blocking conductor 9 corresponds to the temperature of the evaporator ceramic 6. The at least one blocking conductor 9 is configured in such a manner that when the operation end temperature is exceeded, it exhibits an abruptly increasing electrical resistance. Below the operation end temperature, the at least one blocking conductor 9 is thus electrically conductive, so that the evaporator ceramic 6 upon electrical supply is operated in the operating range, i.e. reaches temperatures up to the operation end temperature. The abrupt increase of the electrical resistance of the at least one blocking conductor 9 results in that the electric current flowing through the evaporator ceramic 6, upon exceeding of the operation end temperature, is interrupted or substantially reduced so that the abrupt increase of the electrical resistance of the at least one blocking conductor 9 defines or at least dominates the operation end temperature. It is thus possible to operate the evaporator device 2 with controlled evaporation parameters. This allows in particular evaporating a predetermined quantity of the liquid to be evaporated and thus a predetermined dose of the liquid. For this purpose, a separate electronics (not shown) and/or a separate sensorics (not shown) for example for determining the temperature of the evaporator ceramic 6 are not necessary. Accordingly, the inhaler 1 of the shown exemplary embodiments advantageously comprises no such sensorics and electronics.

As is evident in particular from the FIGS. 2 and 3, the electrical connections 5 shown in the exemplary embodiments are each formed as a circuit board 10, for example of a metal or of a metal alloy.

In the exemplary embodiment shown in the FIGS. 2 and 3, the evaporator 4 is arranged between the connections 5. In the exemplary embodiments shown in the FIGS. 2 and 3, the evaporator 4 is arranged between the connections 5. In the exemplary embodiment shown in the FIGS. 4 and 5, both connections 5 are arranged on the same end face of the evaporator 4.

In the shown exemplary embodiments, the evaporator 4, in particular the evaporator ceramic 6, and the at least one blocking conductor 9 form a contiguous module 11, which is arranged between the connections 5. In the exemplary embodiment shown in the FIGS. 2 and 3, the module 11 has a cuboid form.

In the exemplary embodiment shown in FIG. 2, the evaporator ceramic 6 is formed so as to be contiguous and cuboid, wherein between the respective outer side of the evaporator ceramic 6 facing one of the connections 4 and the associated connection 5 a blocking conductor 9 is arranged.

The exemplary embodiment shown in FIG. 3 differs from the exemplary embodiment shown in FIG. 1 in that the evaporator ceramic 6 is formed in two parts and thus comprises two evaporator bodies 12, which in the shown exemplary embodiment are each formed identically and cuboid in shape. In the shown exemplary embodiment, a single blocking conductor 9 is provided, which is arranged between the evaporator bodies 12.

In the exemplary embodiment shown in the FIGS. 4 and 5, the evaporator 4, in particular the evaporator ceramic 6, is formed annular in shape. There, the evaporator 4 is formed integrally.

As is evident in particular from the FIGS. 1 and 3, a volume portion of the evaporator ceramic 6 in the total volume of the module 11 is substantially larger in the shown exemplary embodiments than a volume portion of the at least one blocking conductor 9. In particular, the volume portion of the at least one blocking conductor 9, in the following also referred to as blocking volume, is maximally 1/10 of the volume portion of the evaporator ceramic 6, in the following also referred to as evaporator volume. This results in particular in that the total volume for receiving the liquid is defined or at least dominated by the evaporator ceramic 6. This results in that the at least one blocking conductor 9 in the operating range plays a negligible role in the electrical total resistance of the module 11 and thus of the evaporator 4. In other words, the electrical total resistance of the evaporator 4 in the operating range is dominated by the evaporator ceramic 6, whereas above the operating range it is dominated by the at least one blocking conductor 9.

In the shown exemplary embodiments, the respective blocking conductor 9, compared with the evaporator ceramic 6 or the evaporator bodies 12, is formed as a thin layer 13 and can therefore be also referred to as blocking layer 14.

The respective blocking conductor 9 is preferentially a PTC thermistor 15, which from a starting temperature has an electrical resistance that abruptly rises by multiple powers of ten. Here, the operation end temperature advantageously corresponds to a temperature between the starting temperature and an end temperature of the PTC thermistor 15, in particular the starting temperature of the PTC thermistor 15.

In particular, the PTC thermistor 15 is a ceramic 16 differing from the evaporator ceramic 6, which in the following is also referred to as blocking ceramic 16. Because of the lower blocking volume of the blocking ceramic 16 compared with the evaporator volume of the evaporator ceramic 6, the total capacity of the evaporator 4 is defined or at least dominated by the evaporator ceramic 6.

In the shown exemplary embodiments and preferably the evaporator device 2 and the container 3 form a unit 25, which is replaceably received in the inhaler 1, in particular in the housing 17. The container 3 of the shown exemplary embodiments is sealed. This means that the container 3 cannot be refilled with liquid without the container 3 being damaged, for example drilled open, broken and the like.

Alternatively it is possible that the container 3 is firmly received in the inhaler 1 and refillable. In this case, the evaporator device 2 can also be permanently received in the inhaler 1.

Alternatively it is possible that the container 3 is replaceable. In this case, the evaporator device 2 can also be permanently received in the inhaler 1.

The inhaler 1 of the shown exemplary embodiments is designed in such a manner that the control device 21 communicates with the container 3 and/or the evaporator device 2, in order to detect and/or receive in particular the liquid to be evaporated and/or the received unit 25. For this purpose, the control device 21 comprises a communications interface 36 merely shown in FIG. 1, which in the following is also referred to as control device communications interface 36. In the shown exemplary embodiments, the unit 25, further, comprises a communications interface 37, which in the following is also referred to as unit communications interface 37. Control device communications interface 36 and unit communications interface 37, in the state of the unit 25 received in the inhaler 1, are communicatingly interconnected, preferentially communicatingly interconnected wirelessly. The unit communications interface 37 and the control device 21, in particular the control device communications interface 36, are configured in such a manner that the control device 21 detects or receives the unit 25 and/or the liquid. For this purpose, the unit communications interface 37 can contain corresponding information. If no approved unit 25 and/or no unit 25 of a predetermined kind is received in the inhaler 1, the control device 21 is practically configured in such a manner that it prevents an operation of the inhaler 1. Likewise, the control device 21 can be configured in such a manner that it prevents an operation of the inhaler 1 upon missing communication with the unit 25.

In the shown exemplary embodiments, the adjusting of the inhaler 1 between the filling state 23 and the evaporation state 24 takes place by way of a relative movement between the evaporator device 2, in particular of the evaporator ceramic 6, and the container 3. In the shown exemplary embodiments, this relative movement takes place translationally or linearly. Rotational relative movements between the evaporator device 2 and the container 3 are also conceivable. In the shown exemplary embodiments, the relative movement between the evaporator device 2, in particular the evaporator ceramic 6, and the container 3 takes place by a relative movement of the evaporator device 2 to the container 3.

In the exemplary embodiment shown in the FIGS. 4 and 5, the container 3 has a cylindrical shape with an inner surface 27 delimiting a hollow space 26 and an outer surface 28 facing away from the inner surface 27. The inner surface 27 and the outer surface 28 delimit the container volume 22. The evaporator device 2 is arranged in the hollow space 26 and guided in the manner of a piston along the inner surface 27 and thus adjustable. For this purpose, the inner surface 27 has a corresponding inner contour 29. The container 3 comprises, on the inner surface 27, in particular the inner contour 29, an open outlet 30, in the following also referred to as container outlet 30. The container outlet 30 serves for letting out the liquid stored in the container volume 22 into the receiving structure 7. Here, the receiving structure 7 comprises a surface 31 facing the inner surface 27, via which in the filling state 23 liquid, via the container outlet 30, enters the receiving structure 7. The receiving structure 7, in the shown exemplary embodiment, comprises, spaced apart from this surface 31, which in the following is also referred to as receiving surface 31, a surface 32 for dispensing the liquid evaporated in the evaporation state 24. This surface 32 is also referred to as dispensing surface 32 in the following. In the exemplary embodiment shown in the FIGS. 4 and 5, the receiving surface 31 and the dispensing surface 32 face away from one another. The dispensing surface 32 is thus arranged on the side of the receiving surface 31 facing away from the inner surface 27 of the container 3. The dispensing surface 32 is open towards the hollow space 26. In the evaporation state 24, the receiving surface 31, preferentially the receiving structure 7 is spaced apart from the container outlet 30. Further, the evaporator device 1 comprises a seal 33 which is arranged on an end face of the receiving structure 7 or of the evaporator ceramic 6. This seal 33, which in the following is also referred to as first seal 33a, seals, in the evaporation state 24, the container outlet 30 and fluidically seals the container outlet 30. Compared with this, the receiving surface 31, in the filling state 23, adjoins the container outlet 30.

In the exemplary embodiment shown in the FIGS. 4 and 5, the evaporator device 2 has a further seal 33 arranged on the end face on the side of the receiving structure 7 or the evaporator ceramic 6 facing away from the first seal 33a, which in the following is also referred to as second seal 33. The second seal 33b prevents in particular vapour exiting from the end face of the receiving structure 7, which it covers. Both seals 33, further, prevent that liquid directly enters the hollow space 26 via the container outlet 30.

In the exemplary embodiment shown in the FIGS. 4 and 5, the container 3, in particular the inner surface 27, forms a chimney 34 for the evaporated liquid. For this purpose, the container 3 has a cross-section that can be flowed through, which decreases towards the outlet opening 18. In the shown exemplary embodiment, the container 3 has a constant cross-section up to a stop 35, which from the stop 35 is decreased towards the outlet opening 18. As is evident in particular from FIG. 5, the stop 35 in the shown exemplary embodiment forms a limitation for the evaporator device 2 in the evaporation state. Here, the second seal 33b in the evaporation state 24 a butts up against the stop 35.

1. An inhaler for inhaling an aerosol, comprising:
a container configured to store a liquid;
an evaporator device including an electric evaporator configured to evaporate the liquid to produce the aerosol;
the evaporator device further including a receiving structure having a predetermined total volume for receiving the liquid; and
a controller;
wherein the inhaler is adjustable to a filling state and to an evaporation state;
wherein, in the filling state, the receiving structure and the container are fluidically connected to one another such that the liquid stored in the container is flowable into the receiving structure;
wherein, in the evaporation state, the receiving structure and the container are fluidically separated from one another such that the liquid is not flowable from the container into the receiving structure; and
wherein the controller is configured to electrically supply the evaporator in the evaporation state such that the liquid received in the receiving structure evaporates.

2. The inhaler according to claim 1, wherein the controller is further configured to interrupt the electrical supply of the evaporator in the filling state.

3. The inhaler according to claim 1, wherein the controller is further configured to prevent an adjusting from the evaporation state to the filling state until the liquid received in the receiving structure is completely evaporated.

4. The inhaler according to claim 1, wherein, in the filling state, the receiving structure is completely filled with the liquid stored in the container.

5. The inhaler according to claim 1, wherein the evaporator includes the receiving structure.

6. The inhaler according to claim 5, wherein the evaporator is an electrically conductive evaporator ceramic.

7. The inhaler according to claim 6, further comprising (i) at least one blocking conductor and (ii) two electrical connections for an electrical supply of the evaporator ceramic, wherein:
the evaporator ceramic is configured to, during operation and upon electrical supply, homogeneously provide heat in a thermal operating range between an operation starting temperature and an operation end temperature for evaporating the liquid received in the receiving structure;
an electrical current path for the electrical supply of the evaporator ceramic extends through the two electrical connections and through the evaporator ceramic;
the at least one blocking conductor is arranged in the current path and is heat-transmittingly connected to the evaporator ceramic; and
the at least one blocking conductor is configured such that when the operation end temperature is exceeded an electrical resistance of the at least one blocking conductor abruptly increases.

8. The inhaler according to claim 1, wherein the evaporator device and the container, for adjusting between the filling state and the evaporation state, are moveable relative to one another.

9. The inhaler according to claim 8, wherein:
the container includes a container outlet;
the evaporator device further includes a seal adjoining a receiving surface of the receiving structure;
the receiving surface, in the filling state, adjoins the container outlet and the receiving structure and is fluidically connected to the container; and
the seal seals the container outlet in the evaporation state.

10. The inhaler according to claim 9, wherein:
the receiving structure includes a dispensing surface disposed spaced apart from the receiving surface; and
in the evaporation state and upon electrical supply of the evaporator, the liquid evaporated in the receiving structure exits from the dispensing surface.

11. The inhaler according to claim 9, wherein:
the container further includes an inner contour, which includes the container outlet; and
the evaporation device, for adjusting between the filling state and the evaporation state, is guided along the inner contour.

12. The inhaler according to claim 11, wherein the container is structured as a cylinder in which the evaporator device is guided in the manner of a piston.

13. The inhaler according to claim 11, wherein the container forms a chimney for the liquid evaporated with the evaporator.

14. The inhaler according to claim 1, wherein the receiving structure includes a plurality of pores.

15. A replaceable unit for the inhaler according to claim 1, the replaceable unit comprising the container and the evaporator device.

16. The unit according to claim 15, further comprising a unit communications interface which, in a state received in the inhaler, is communicatingly connected to a controller communications interface of the controller.

17. The inhaler according to claim 1, wherein the receiving structure is configured as a pore structure.

18. An inhaler for inhaling an aerosol, comprising:
a container configured to store a liquid;
an evaporator including:
an evaporator ceramic configured to evaporate the liquid to produce the aerosol; and
a receiving structure having a predetermined total volume for receiving the liquid;
a controller configured to selectively supply electricity to the evaporator ceramic;
